(19) United States
(12) Patent Application Publication
(10) Pub. No.: US 2025/0009509 A1
(43) Pub. Date: Jan. 9, 2025

(54) FUNCTIONAL POLYMER NANOCOMPOSITES FOR STIMULI-RESPONSIVE DYNAMIC RING TO TREAT CARDIAC MITRAL VALVE DISORDER

(71) Applicant: Qatar Foundation for Education, Science and Community Development, Doha (QA)

(72) Inventors: Muammer Koc, Doha (QA); Ans Al Rashid, Doha (QA); Hamid Ikram, Doha (QA); Dawoud I.A. Al Kindi, Doha (QA)

(21) Appl. No.: 18/762,055

(22) Filed: Jul. 2, 2024

Related U.S. Application Data
(60) Provisional application No. 63/525,285, filed on Jul. 6, 2023.

(51) Int. Cl. A61F 2/24; A61L 27/04; A61L 27/16; A61N 2/00

(52) U.S. Cl. CPC A61F 2/2445; A61L 27/042; A61L 27/16; A61N 2/004

(57) ABSTRACT

Example systems, methods, and apparatus are disclosed herein for functional polymer nanocomposites for stimuli-responsive Dynamic Ring to treat Cardiac Mitral Valve Disorder. The stimuli-responsive material may be a magnetically-induced shape memory nanoparticle such as Hematite dispersed in PLA. Such Hematite may be at a concentration of 10 wt % to 20 wt %. The Dynamic Ring may be produced by a process of additive manufacturing including the polymer nanocomposites in the vicinity of the patient.

### PRIORITY CLAIM AND CROSS-REFERENCE TO RELATED APPLICATIONS

The present application claims the benefit of and priority to U.S. Provisional Application No. 63/525,285 filed Jul. 6, 2023, which is incorporated herein by reference in its entirety.

### BACKGROUND

As of now, heart valve substitutions are perhaps the most broadly utilized prosthetic methods. Statistics have shown that in 2006 there were around 98,000 valve substitutions across the USA and 195,300 from other countries. The aortic valve manages the blood supply to each of the significant vessels in the body, and it needs to be replaced in case of malfunctioning. The deficiency occurs due to the narrowing of blood vessels interfering with smooth blood flow, which compromises the blood retention. Poor retention of the blood in the valve causes it to flow back into the heart resulting in regurgitation of the blood, termed aortic stenosis (AS). Similarly, the mitral valve has the same function as the aortic valve in the left heart chamber. Like aortic stenosis, mitral valve regurgitation (MVR) is also very common and has become a target for researchers and the medical industry. However, transcatheter aortic valve substitution (TAVS) is a procedure in which a catheter is inserted into the leg or chest and guided to the heart for the substitution of a diseased aortic valve with an artificial valve, has turned into the standard consideration for treatment of aortic stenosis. In contrast, mitral valve (MV) substitution has not yet accomplished a similar outcome.

MV issues are more frequent than AS, and the surgical methodology stays the highest quality therapy for degenerative MV regurgitation. The medical treatment is not adequate for patients with an increased risk of surgery due to medical complications and who are unwilling to undergo surgical procedures. Studies have suggested that MV repair should be preferred over its replacement like TAVS for several functional and physiological reasons. The Transcatheter MV replacement (TMVR) procedure is not mature yet, and there are a few surgeries accomplished worldwide. Compared to the TAVS, several challenges are present for the delivery to TMVR, positioning of the valve, and transseptal puncture. The location of the MV is in-between the left atrium and left ventricle, making the delivery procedure difficult as there is no direct access to the valve. Therefore, puncturing the heart from the vessel side or the bottom side that directly opens to MV is followed, which offers an easier way for perfect positioning and repairing the valve but is highly associated with myocardial damage. The mitral valve possesses anterior and posterior leaflets, which open and closes synchronized to ensure the one-way blood flow from the left atrium to the heart's left ventricle. These leaflets are supported by a fibrous ring called the annulus, which ensures the proper positioning with the help of tendineae muscles. Similarly, replacing the MV valve with a fabricated valve made of metal and composites is adopted with open heart surgeries but carries a high risk.

Although metallic MV replacements provide the desired mechanical properties, clinical issues related to these materials led to the exploration of polymers as potential materials. Prime shortcomings associated with polymers are lack of radiopacity, lower radial stresses, and plasticity. As polymers exhibit inferior mechanical properties, thicker struts can avoid elastic recoil and withstand radial stresses. Poly L-lactic acid (PLLA), FDA approved biomaterial, has been utilized recently to serve the cause. Several attempts have been made to fabricate MV replacements using different polymeric and composite materials, including; polylactic acid (PLA) with chitosan and paclitaxel coating, polyurethane (PU) added to polyester, PLLA, polycaprolactone (PCL) with wireless pressure sensors. PLA is a thermoplastic polyester obtained from biodegradable resources and exhibits excellent mechanical and thermal properties, good processability, and low environmental impact. PLA is globally accepted as biodegradable because it originates from plants and has proven safe for biomedical applications and food industries. Therefore, being explored in tissue engineering, biomedical implants, and food packaging sectors for their performance.

In addition, MV replacements made of high-paramagnetic iron oxide nanoparticles encourage functionalized intervention devices for different biomedical applications. High-paramagnetic nanoparticles that are common in the research community are magnetite (Fe3O4) and hematite (α-Fe2O3), which are usually coated with polymers. The coating is used for enhanced dispersion of the particles in the mixture for stable samples, allowing the conjugation of magnetic-sensitive ligands and increasing the magnetization properties. Moreover, as magnetite and hematite display high paramagnetic properties, the remote actuation of their mass structure is conceivable through an external magnetic field for cardiovascular therapy.

Magnetic-sensitive nanoparticles have been widely adopted for various applications, especially biomedical (drug delivery, stimuli-responsive stents, and magnetic resonance imaging), storage media, and photochemical applications. Specifically, hematite (α-Fe2O3) is one of the most common magnetic sensitive oxides which possess unique electrical and magnetic properties relying upon the Fe+2 ion. Due to their attractive structural, chemical, thermal and suitable surface characteristics, superparamagnetic nanoparticles are being explored for biomedical applications. Several methods are available to synthesize various nanocomposites with desired properties. However, the technique used for biomedical applications, especially functional cardiovascular stents, is still under development. The interest in metal nanoparticle research is increasing nowadays, especially in plant tissues, plant byproducts, and other parts of biological plants. The magnetic behavior of PLA/Fe2O3 is studied by Zhang et al., “Design of 4D Printed Shape-Changing Tracheal Stent and Remote Controlling Actuation” (2021), which is hereby incorporated by reference in its entirety, where the shape-changing behavior of the composites with time was investigated, which ultimately satisfies the shape-changing properties of polymer composites with time.

Therefore, MV repair is still under clinical development and needs to be addressed with novel solutions. These processes have proved their potential in recent years over many applications. Stimuli-responsive dynamic rings are required that can be actuated remotely through a magnetic field and adjusted according to the requirements.

### SUMMARY

Example systems, methods, and apparatus are disclosed herein for functional polymer nanocomposites for stimuli-responsive Dynamic Ring to treat Cardiac Mitral Valve Disorder.

The disclosed invention proposes a novel approach to repairing the MV by installing a stimuli-responsive dynamic ring above the annulus. The patient-specific dynamic ring (depending upon the patient's age, physiological conditions, etc.) will be manufactured via an advanced manufacturing process (for rapid and customized fabrication) using biocompatible and cytocompatible polymers reinforced with magnetic sensitive nanoparticles to provide the stimulus on exposure to the magnetic field. The dynamic ring will reposition the annulus with a proper diameter by actuating it remotely, which will situate the MV in a proper working position with no leakage instead of replacing the whole valve with a fabricated one, eliminating surgeries and puncturing of the heart.

To achieve the facile fabrication of complex dynamic ring structures with minimal waste and facilitate rapid fabrication, 3D printing (3DP) processes are considered herein. As seen in the following disclosure, stimuli-responsive polymer nanocomposites were synthesized by doping α-Fe2O3 into polylactic acid (PLA) through a solvent-casting approach. The nanocomposites were then characterized using Fourier transform infrared spectroscopy (FTIR), thermogravimetric analysis (TGA), and differential scanning calorimetry (DSC). Finally, electromagnetic capability using magnetic hysteresis analysis is also performed to assess actuation under magnetic stimulus in a controlled environment.

In light of the disclosure herein, and without limiting the scope of the invention in any way, in a first aspect of the present disclosure, which may be combined with any other aspect listed herein unless specified otherwise, there are provided functional polymer nanocomposites for stimuli-responsive dynamic ring to treat cardiac mitral valve disorder.

In a second aspect of the present disclosure, which may be combined with any other aspect listed herein unless specified otherwise, there is provided a dynamic ring to treat cardiac mitral valve disorder comprising a magnetic nanoparticle configured to enable magnetically induced shape memory effects.

In a third aspect of the present disclosure, which may be combined with any other aspect listed herein unless specified otherwise, the magnetic nanoparticle comprises Hematite (α-Fe₂O₃).

In a fourth aspect of the present disclosure, which may be combined with any other aspect listed herein unless specified otherwise, the concentration of Hematite is at least 10 wt %.

In a fifth aspect of the present disclosure, which may be combined with any other aspect listed herein unless specified otherwise, the concentration of Hematite is at least 15 wt %.

In a sixth aspect of the present disclosure, which may be combined with any other aspect listed herein unless specified otherwise, the concentration of Hematite is at least 20 wt %.

In a seventh aspect of the present disclosure, which may be combined with any other aspect listed herein unless specified otherwise, the dynamic ring is configured to have a desired magnetically induced shape memory position for a mitral valve replacement treatment.

In an eighth aspect of the present disclosure, which may be combined with any other aspect listed herein unless specified otherwise, there is provided a method of fabricating functional polymer nanocomposites for stimuli-responsive dynamic ring to treat cardiac mitral valve disorder.

In a ninth aspect of the present disclosure, which may be combined with any other aspect listed herein unless specified otherwise, there is provided a method of fabricating a patient-specific stimuli-responsive dynamic ring to treat cardiac mitral valve disorder comprising dissolving PLA in dichloromethane (DCM) using a mechanical mixer; adding a concentration of magnetic sensitive α-Fe2O3 nanoparticles to the PLA/DCM solution; mechanically stirring the solution for 4 hours at a speed of between 1300-1500 rpm; heating the solution to 40° C. with continuous stirring to remove the solvent (DCM) from the mixture; laying the PLA/α-Fe2O3 nanocomposites on a flat surface to remove the entrapped solvent; and obtaining nanocomposites in film form.

In another aspect of the present disclosure, which may be combined with any other aspect listed herein unless specified otherwise, the method further comprises forming a dynamic ring by additive manufacturing using the nanocomposites.

In another aspect of the present disclosure, which may be combined with any other aspect listed herein unless specified otherwise, the concentration of magnetic sensitive Hematite (α-Fe2O3) is 10 wt %.

In another aspect of the present disclosure, which may be combined with any other aspect listed herein unless specified otherwise, the concentration of magnetic sensitive Hematite (α-Fe2O3) is 15 wt %.

In another aspect of the present disclosure, which may be combined with any other aspect listed herein unless specified otherwise, the concentration of magnetic sensitive Hematite (α-Fe2O3) is 20 wt %.

In another aspect of the present disclosure, which may be combined with any other aspect listed herein unless specified otherwise, the method further comprises configuring the dynamic ring to have a desired magnetically induced shape memory position.

In another aspect of the present disclosure, which may be combined with any other aspect listed herein unless specified otherwise, there is provided a method of using functional polymer nanocomposites for stimuli-responsive dynamic ring to treat cardiac mitral valve disorder.

In another aspect of the present disclosure, which may be combined with any other aspect listed herein unless specified otherwise,

In another aspect of the present disclosure, which may be combined with any other aspect listed herein unless specified otherwise, there is provided a method of using a stimuli-responsive dynamic ring to treat cardiac mitral valve disorder comprising: determining a desired configuration of the stimuli-responsive dynamic ring based on a physiology of a patient; and producing a stimuli-responsive dynamic ring based on the desired configuration.

In another aspect of the present disclosure, which may be combined with any other aspect listed herein unless specified otherwise, the dynamic ring is produced by a process of additive manufacturing.

In another aspect of the present disclosure, which may be combined with any other aspect listed herein unless specified otherwise, the process of additive manufacturing occurs within the vicinity of the patient.

In another aspect of the present disclosure, which may be combined with any other aspect listed herein unless specified otherwise, the dynamic ring comprises a PLA/α-Fe2O3 nanocomposite.

In another aspect of the present disclosure, which may be combined with any other aspect listed herein unless specified otherwise, the concentration of α-Fe₂O₃ in the PLA/α-Fe₂O₃ nanocomposite is at least 10 wt %.

In another aspect of the present disclosure, which may be combined with any other aspect listed herein unless specified otherwise, the concentration of α-Fe₂O₃ in the PLA/α-Fe₂O₃ nanocomposite is at least 15 wt %.

In another aspect of the present disclosure, which may be combined with any other aspect listed herein unless specified otherwise, the concentration of α-Fe₂O₃ in the PLA/α-Fe₂O₃ nanocomposite is at least 20 wt %.

In another aspect of the present disclosure, which may be combined with any other aspect listed herein unless specified otherwise, the method further comprises providing the dynamic ring to the patient; and actuating the dynamic ring by applying a magnetic field near the dynamic ring.

In another aspect of the present disclosure, any of the structure, functionality, and alternatives disclosed in connection with any one or more of FIGS. 1 to 4 may be combined with any other structure, functionality, and alternatives disclosed in connection with any other one or more of FIGS. 1 to 4.

In light of the present disclosure and the above aspects, it is therefore an advantage of the present disclosure to provide users with functional polymer nanocomposites for stimuli-responsive Dynamic Ring to treat Cardiac Mitral Valve Disorder.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. In addition, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a graph of FTIR spectrum of PLA/α-Fe2O3 Nanocomposites, according to an example embodiment of the present disclosure.

FIG. 2 is a graph of DSC analysis of PLA/α-Fe2O3 Nanocomposites, according to an example embodiment of the present disclosure.

FIG. 3 is a graph of TGA analysis of PLA/α-Fe2O3 Nanocomposites, according to an example embodiment of the present disclosure.

FIG. 4 is a graph of Magnetic Sensitivity of PLA/α-Fe2O3 Nanocomposites, according to an example embodiment of the present disclosure.

### DETAILED DESCRIPTION

Methods, systems, and apparatus are disclosed herein for functional polymer nanocomposites for stimuli-responsive Dynamic Ring to treat Cardiac Mitral Valve Disorder.

While the example methods, apparatus, and systems are disclosed herein functional polymer nanocomposites for stimuli-responsive Dynamic Ring to treat Cardiac Mitral Valve Disorder, it should be appreciated that the methods, apparatus, and systems may be operable for other medical conditions.

The disclosed invention presents preliminary findings on the performance of the stimuli-responsive PLA/α-Fe2O3 nanocomposites under magnetic stimulus with varying concentrations of hematite (α-Fe2O3) nanoparticles. The effect of varying nanoparticle concentrations on the structural and thermal properties was examined using Fourier transform infrared spectroscopy (FTIR), thermogravimetric analysis (TGA), and differential scanning calorimetry (DSC). Subsequently, the magnetic field with various intensities was generated using a vibrating sample magnetometer in a controlled environment. The resulting deformation rate of the nanocomposites was observed in response to changing magnetic fields. The results revealed that the PLA/α-Fe2O3 nanocomposites could be potential materials for biomedical applications, i.e., cardiovascular stents, which can provide the desired stimulus to treat post-stenting complications without the need for secondary surgical procedures. In future studies, the synthesized PLA/α-Fe2O3 nanocomposites will be adopted for 3D printing (3DP) processes to develop patient-specific cardiovascular stents and to evaluate their biomechanical performance.

The stimuli-responsive dynamic ring may be produced by a process of 3DP on-site at a health care facility based on a measured physiology of a patient. For example, the diameter of a specific annulus in the vasculature of a patient may be measured and used to determine the dimensions of the dynamic ring produced by 3DP. Such a system and method may be advantageous when supply chain restrictions cause a delay or lack of available TAVS or MV substitutes. Another advantage to the systems and methods is increased efficaciousness because of the customized patient-specific dimensions of the dynamic rings. Another advantage is the resilience of the dynamic rings based on their shape-memory characteristics and magnetic actuation. Certain concentrations of shape-memory nanoparticle may be advantageous based on the specific needs of a patient, location of the valve, the strength of the magnetic field used to actuate the cardiovascular stent, and any other possible variables.

### Preparation and Materials

Synthesis of PLA α-Fe2O3 Nanocomposite: The solvent casting approach was utilized to synthesize the polymer nanocomposites. First, PLA pallets were dissolved in dichloromethane (DCM) using a mechanical mixer. Varying concentrations (10 wt %, 15 wt %, and 20 wt %) of magnetic sensitive α-Fe2O3 nanoparticles were added to the PLA/DCM solution and mechanically stirred for 4 hours at a speed of 1300-1500 rpm. Subsequently, the solution was heated to 40° C. with continuous stirring to remove the solvent (DCM) from the mixture. Finally, the PLA/α-Fe2O3 nanocomposites were laid on a flat surface to remove the entrapped solvent, and nanocomposites were obtained in film form.

PLA α-Fe2O3 Nanocomposite Characterization: FTIR analysis was performed in transmittance mode to gain chemical insights into the stimuli-responsive nanocomposites using (Thermo Scientific Nicolet iS50 FT-IR) spectrometer equipped with an attenuated total reflectance (ATR) sampling accessory with a diamond crystal plate. The thermal properties of the nanocomposite were characterized using thermogravimetric analysis (TGA) and differential scanning calorimetry (DSC). For TGA, the analyzer STA7300 (from Hitachi company) was used, with the temperature range from room temperature to 700° C. at a rate of 10° C./min under a nitrogen gas environment to avoid oxidation. For DSC, the SF1942 instrument (from Mettler Toledo company, Switzerland) was used at a temperature range from room temperature to 200° C. at a rate of 10° C./min.

### Magnetic Stimuli Response

Using a magnetic field, the PLA/α-Fe2O3 nanocomposites were also characterized for their response under remote actuation. A vibrating sample magnetometer (VSM, Lakeshore 340, USA) provided the required magnetic intensity at room temperature (300 K) by saturating the sample in a magnetic field of strength 8T. The PLA/α-Fe2O3 nanocomposites were converted to powder form with an average particle size of 50-60 μm to be fed into the manometer.

### Fourier Transform Infrared Spectroscopy (FTIR)

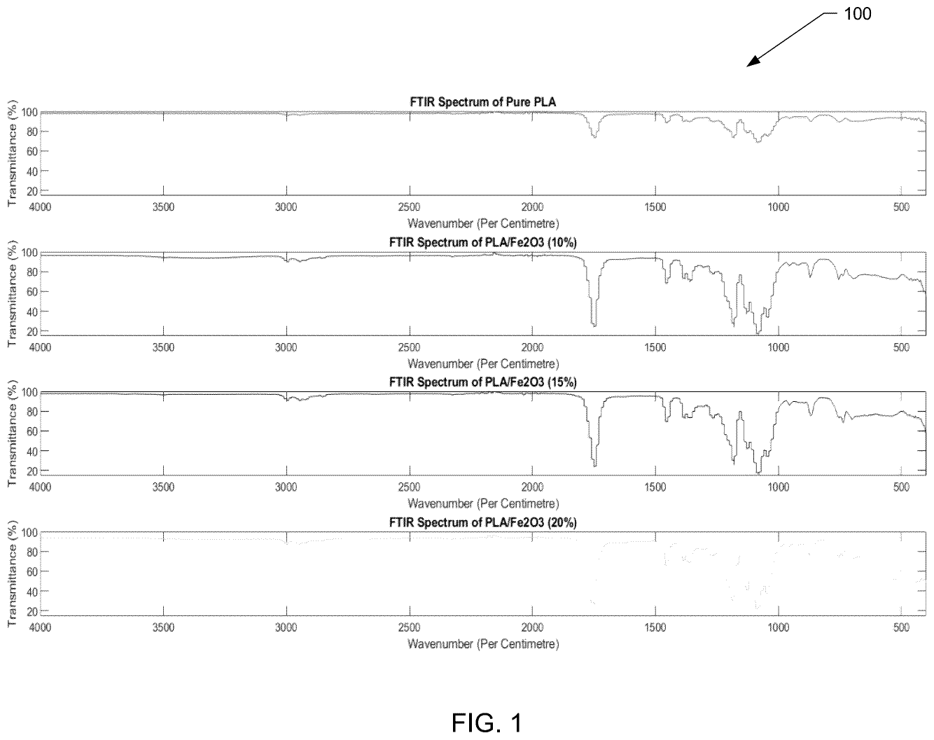

FTIR analysis can provide significant insight into the structure of polymer nanocomposites by identifying the molecular interactions. Considerable changes (band shifting or broadening) can be observed between pure polymer and polymer nanocomposite spectra in the case of chemical interactions (i.e., hydrogen bonding or dipolar interaction). A characteristic peak at a lower wavenumber corresponds to a strong interaction between polymer and nanoparticles. FIG. 1 shows the FTIR spectra of pure PLA and synthesized PLA-reinforced hematite (α-Fe2O3) nanoparticles 100. Three characteristic peaks were observed for pure PLA, two between 1000 cm-1 to 1250 cm-1 and one around 1750 cm-1, corresponding to stretching of —OH, —C═O, and C—O groups. For PLA/α-Fe2O3 nanocomposites, the stretching in peaks between 1000 cm-1 to 1250 cm-1 and 1750 cm-1 is noticeable due to the bending vibration of hydroxyl groups and C═O stretching vibration, respectively. Similar effects were also observed by incorporating carbon black and carbon nanotubes into PLA. Finally, the characteristic peak for α-Fe2O3 nanoparticles at around 3000 cm-1 was not reasonably detected due to the low concentration of α-Fe2O3 nanoparticles within the PLA.

### DSC & TGA Analysis

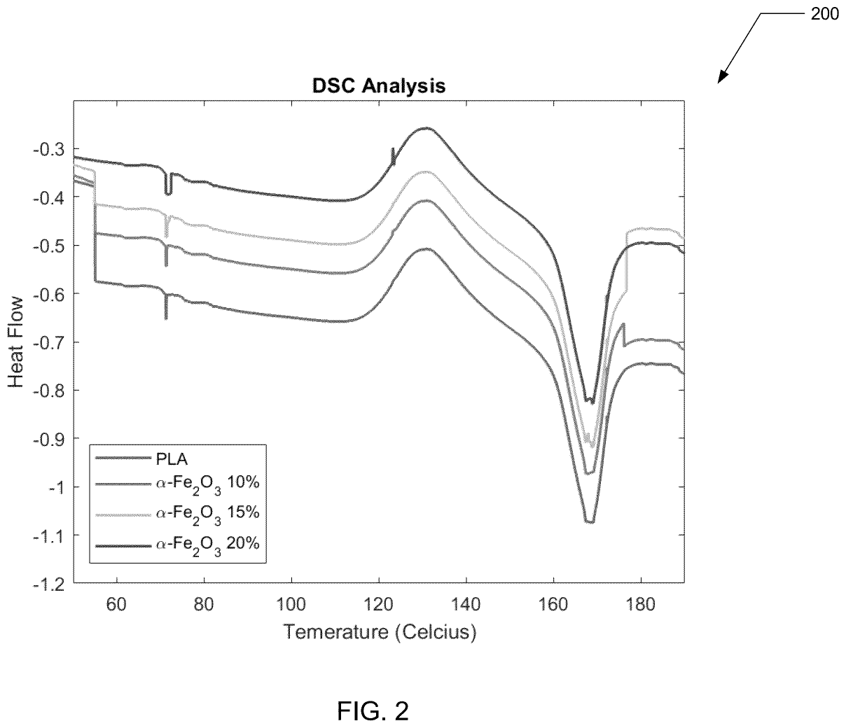

The stimuli-responsive PLA/α-Fe2O3 nanocomposites were characterized using DSC and TGA. FIG. 2 is a graph of DSC analysis of PLA/α-Fe2O3 Nanocomposites DSC results 200. FIG. 2 shows that at around 71° C., there is a sharp valley in the curve, indicating the glass transition temperature of the compound (and the shape that the shape memory would be if it were 3D-printed). The slope of the curve begins to increase at about 110° C., indicating the crystallization of the PLA/α-Fe2O3 polymer nanocomposites. The gradient next takes a steep hit starting at about 153° C., showing that the melting point of the composites has been reached. However, the heat flow is increased due to the reinforcement of α-Fe2O3 to PLA. The first drop in heat flow at 71.38° C. for pure PLA corresponds to the glass transition temperature. In addition, a significant increase in the slope is observed at 131° C., referring to the crystallization temperature. At the melting temperature, there is a substantial drop in the heat flow, and the drop at this point is consistent for all the materials under consideration. DSC curves of all PLA/α-Fe2O3 concentrations have produced the same pattern, showing that the addition of α-Fe2O3 does not have a significant effect on these temperatures, although the heat flow is increased by almost 54% with 20% reinforcement. The glass transition, crystallization, and melting temperatures of each material are reported in Table 1 (below).

TABLE 1
Phase change temperature of PLA/α-Fe2O3 Nanocomposites.

| | Glass Transition | Crystallization | Melting |
|---|---|---|---|
| PLA/Composite | Temperature (° C.) | Temperature (° C.) | Temperature (° C.) |
| Pure PLA | 71.38 | 131.00 | 168.66 |
| PLA/α-Fe₂O₃10% | 72.34 | 132.32 | 169.4 |
| PLA/α-Fe₂O₃15% | 73.2 | 133.4 | 170.3 |
| PLA/α-Fe₂O₃20% | 74 | 134.1 | 171.2 |

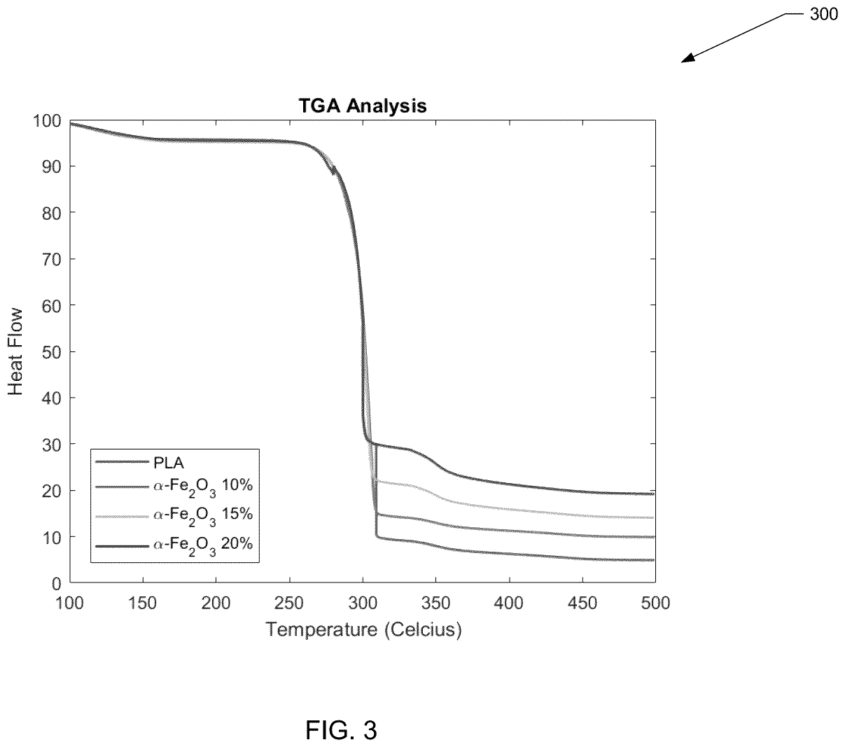

The thermal stability of the samples was analyzed using TGA. TGA was performed between 0° C. to 500° C. at a temperature ramp of 10° C./min. FIG. 3 is a graph of TGA analysis of PLA/α-Fe2O3 Nanocomposites 300. FIG. 3 shows the results for pure PLA and PLA/α-Fe2O3 nanocomposites with increasing weight composition (10%, 15%, and 20%), respectively. As seen in the TGA curves, there is a gradual mass loss followed by a steep loss starting at 280° C. This abrupt mass loss is attributed to the loss of PLA from the polymer nanocomposites due to heating. The samples begin to degrade at around 309° C. The melting temperatures of all the PLA/α-Fe2O3 composites increase with increasing composition and as compared with the neat PLA. This is in line with the observation that the melting point of the mixture would be higher than that of the pure substance. Similar DSC and TGA results were observed in the literature by incorporating metallic nanoparticles into the PLA matrix.

### Magnetic Stimuli Response

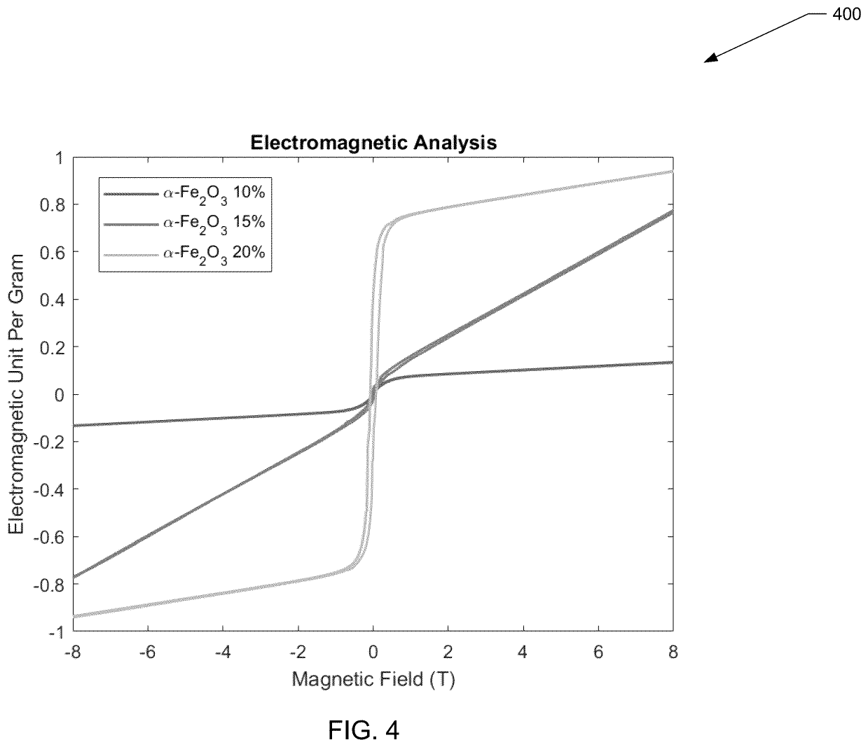

Electromagnetic capability analysis of the synthesized PLA/α-Fe2O3 nanocomposites was also performed to produce magnetic hysteresis curves, as shown in FIG. 4. FIG. 4 is a graph of Magnetic Sensitivity of PLA/α-Fe2O3 Nanocomposites 400. These curves can be quantified using the electromagnetic unit per gram, which indicates the electromagnetic abilities of the tested compound per gram of its weight. The hysteresis curves revealed that the increase in α-Fe2O3 nanoparticles concentration also increases the electromagnetic capabilities of the PLA/α-Fe2O3 nanocomposites. There is visible hysteresis in the compound with the highest α-Fe2O3 concentration and lesser significance at lower concentrations. The space inside the hysteresis loop indicates the magnetic field required to align the ferromagnetic particles with a magnetic field when an external magnetic field is applied. Thus, the maximum force for magnetization is needed for PLA/α-Fe2O3 nanocomposites at 20% of α-Fe2O3 concentration. In addition, there is a more significant electromagnetic capability increase when the concentration of α-Fe2O3 is increased from 15% to 20%, compared to an increase from 10% to 15%. However, the hysteresis curves exhibit properties of soft ferromagnets—i.e., low remanence, lesser coercive force, and low hysteresis losses. Though iron oxide in the mass structure has ferromagnetic characteristics, it also shows superparamagnetic behavior for single-area nanoparticles at the micro and nanoscale. For example, the movement of the sensitive iron oxide particles can easily be adjusted by a remotely applied magnetic field, which generates a net magnetism of the sample. The sample no longer has its polarization if the applied attractive field is turned off.

The disclosed invention uses the Hematite (α-Fe2O3) nanoparticles at different concentrations to synthesize the magnetic responsive PLA/α-Fe2O3 nanocomposites. The PLA/α-Fe2O3 nanocomposites were characterized for their thermochemical and magnetic properties. The results reveal increased heat flow characteristics with the addition of α-Fe2O3 nanoparticles to PLA. The magnetic stimuli response of the PLA/α-Fe2O3 nanocomposites showed that the reinforcement of Hematite (α-Fe2O3) nanoparticles significantly affects the PLA's magnetic properties. The magnetic capability of PLA/α-Fe2O3 nanocomposites demonstrated a drastic increase from 15% to 20%, with an approximate value of 0.82 emu/gram. The reported modification of PLA with α-Fe2O3 nanoparticles suggested the likelihood of directing future work into further analysis of applications where remote actuation is desired. Further studies are required to ensure the effectiveness of its application in the biomedical field by designing and 3D printing of remotely actuated devices to evaluate their deformation and recovery against the magnetic field.

### CONCLUSION

The term vicinity is used herein to describe a relationship between elements. Vicinity may be interpreted as with the same room, within the same building, within the same campus, or any other reasonable interpretation based on the context. For example, and not limiting the scope of any of the claims, the vicinity of a patient in a hospital may reasonably within the same hospital.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A dynamic ring to treat cardiac mitral valve disorder comprising a magnetic nanoparticle configured to enable magnetically induced shape memory effects.

2. The dynamic ring of claim 1, wherein the magnetic nanoparticle comprises Hematite (α-Fe₂O₃).

3. The dynamic ring of claim 2, wherein the concentration of Hematite is at least 10 wt %.

4. The dynamic ring of claim 2, wherein the concentration of Hematite is at least 15 wt %.

5. The dynamic ring of claim 2, wherein the concentration of Hematite is at least 20 wt %.

6. The dynamic ring of claim 1, wherein the dynamic ring is configured to have a desired magnetically induced shape memory position for a mitral valve replacement treatment.

7. A method of fabricating a patient-specific stimuli-responsive dynamic ring to treat cardiac mitral valve disorder comprising:
dissolving PLA in dichloromethane (DCM) using a mechanical mixer;
adding a concentration of magnetic sensitive α-Fe₂O₃ nanoparticles to the PLA/DCM solution;
mechanically stirring the solution for 4 hours at a speed of between 1300-1500 rpm;
heating the solution to 40° C. with continuous stirring to remove the solvent (DCM) from the mixture;
laying the PLA/α-Fe₂O₃ nanocomposites on a flat surface to remove the entrapped solvent; and
obtaining nanocomposites in film form.

8. The method of claim 7, further comprising:
forming a dynamic ring by additive manufacturing using the nanocomposites.

9. The method of claim 8, wherein the concentration of magnetic sensitive Hematite (α-Fe2O3) is 10 wt %.

10. The method of claim 8, wherein the concentration of magnetic sensitive Hematite (α-Fe₂O₃) is 15 wt %.

11. The method of claim 8, wherein the concentration of magnetic sensitive Hematite (α-Fe₂O₃) is 20 wt %.

12. The method of claim 8, further comprising:
configuring the dynamic ring to have a desired magnetically induced shape memory position.

13. A method of using a stimuli-responsive dynamic ring to treat cardiac mitral valve disorder comprising:
determining a desired configuration of the stimuli-responsive dynamic ring based on a physiology of a patient; and
producing a stimuli-responsive dynamic ring based on the desired configuration.

14. The method of claim 13, wherein the dynamic ring is produced by a process of additive manufacturing.

15. The method of claim 14, wherein the process of additive manufacturing occurs within the vicinity of the patient.

16. The method of claim 13, wherein the dynamic ring comprises a PLA/α-Fe₂O₃ nanocomposite.

17. The method of claim 16, wherein the concentration of α-Fe₂O₃ in the PLA/α-Fe₂O₃ nanocomposite is at least 10 wt %.

18. The method of claim 16, wherein the concentration of α-Fe₂O₃ in the PLA/α-Fe₂O₃ nanocomposite is at least 15 wt %.

19. The method of claim 16, wherein the concentration of α-Fe₂O₃ in the PLA/α-Fe₂O₃ nanocomposite is at least 20 wt %.

20. The method of claim 13, further comprising:
providing the dynamic ring to the patient; and
actuating the dynamic ring by applying a magnetic field near the dynamic ring.
